(19) United States
(12) Patent Application Publication
(10) Pub. No.: US 2020/0093744 A1
(43) Pub. Date: Mar. 26, 2020

(54) POLYMER CONSTRUCTS FOR CONTROLLED RELEASE OF GUEST AGENTS

(71) Applicant: CASE WESTERN RESERVE UNIVERSITY, Cleveland, OH (US)

(72) Inventors: Rigoberto Advincula, Cleveland, OH (US); Eric Baer, Cleveland, OH (US); Pengfei Cao, Cleveland, OH (US)

(21) Appl. No.: 16/482,157

(22) PCT Filed: Jan. 30, 2018
(86) PCT No.: PCT/US2018/015933

§ 371 Date: Jul. 30, 2019

Related U.S. Application Data
(60) Provisional application No. 62/452,188, filed on Jan. 30, 2017.

(51) Int. Cl. A61K 9/16; C08G 83/00; C08L 67/04; C08L 79/02

(52) U.S. Cl. CPC A61K 9/1647; C08G 83/005; C08L 67/04; C08L 2203/02; C08L 2207/53; C08L 2201/06; C08L 79/02

(57) ABSTRACT

A polymer construct includes at least one extruded degradable polymer layer having a host polymer material and at least one amphiphilic star-shaped polymer miscible with the host polymer material. The star-shaped polymer includes a polymer core and polymer branches extending therefrom to define a shell around the core. A guest agent is loaded on and/or within the core. The polymer construct upon delivery to a site of interest provides controlled and/or sustained release of the guest agent upon degradation of the at least one polymer layer.

### RELATED APPLICATION

This application claims priority from U.S. Provisional Application No. 62/452,188, filed Jan. 30, 2017, the subject matter of which is incorporated herein by reference in its entirety.

### GOVERNMENT FUNDING

This invention was made with government support under Grant No. DMR-0423914, awarded by The National Science Foundation. The United States government has certain rights to the invention.

### TECHNICAL FIELD

This application relates to extruded polymers and, more specifically, relates to polymer constructs having amphiphilic star-shaped polymers for releasing guest agents in a controlled manner.

### BACKGROUND

Controlled release systems have been widely used in different areas. In agriculture, the controlled release of fertilizer was developed in the 1970s, where sustained and controlled delivery of nutrients following a single application to the soil. In some personal care products, vitamin C and insect repellent lotion can be released in a controlled manner. The main application of the controlled release system is in drug release, especially controlled anticancer drug release.

In the last two decades, rapid advances of nanotechnology catalyzed the transformation of controlled release systems, especially controlled drug delivery, from macro-scale devices to micro and nano-scale systems. To cater to specific needs, the current controlled release systems are mainly polymer-based nano-carriers, in which polymeric nanoparticle and liposomes are dominantly studied. Belonging to the synthetic polyester family, poly(caprolactone) (PCL) is widely used for various biomedical applications due to its good biocompatibility and slow degradation in aqueous environment. Because of the high permeability derived from the rubbery characteristics of PCL, it has been extensively exploited for encapsulation and release of low molecular weight drugs, such as vaccines, steroids and doxorubicin. Due to the hydrophobic and semi-crystalline nature, only the nano-sized PCL devices are used as delivery systems, such as nano-micelles, nano-vesicles and nano-fibers. However, these PCL based nano-devices are rarely employed for industrial applications because of the difficulties in large-scale fabrication.

### SUMMARY

This application describes polymer constructs and methods for fabricating extruded polymer constructs. The polymer constructs include an extruded degradable polymer matrix and amphiphilic star-shaped polymers loaded with one or more guest agents. The amphiphilic star-shaped polymers loaded with one or more guest agents can be uniformly dispersed and/or homogenously distributed within the polymer matrix. The polymer constructs can provide sustained and/or controlled release of the guest agent upon delivery and/or administration of the polymer construct to a site of interest.

Advantageously, the polymer construct can be formed using forced extrusion techniques and the guest agent upon release from the degradable polymer matrix can have the same or substantially similar structural (e.g., size, shape, and morphology), chemical, and/or biochemical (e.g., immune response) characteristics prior to extrusion. Moreover, loading the amphiphilic star-shaped polymer with the guest agent allows guest agents, which are not readily soluble in the polymer matrix by themselves, to be readily loaded and homogenously distributed and/or uniformly dispersed in the polymer matrix.

In some embodiments, where the polymer construct is used for therapeutic applications, the site of interest can be a cell or tissue of a subject. In other embodiments, where the polymer construct is used for agricultural applications, the site of interest can be a plant propagation material, a plant, part of a plant and/or plant organ.

In some embodiments, the polymer construct can be provided in a shape (e.g., a plurality of microparticles) that can be readily delivered to a subject to provide controlled and/or sustained release of the guest agent to cells and/or tissue of a subject. The polymer construct can be administered, injected, or implanted in a minimally invasive fashion in a subject in need thereof to treat diseases (e.g., cancer) and/or disorders in the subject. For example, the polymer constructs can take the form of films, sheets, particles, fibers, etc. on a microscale or nanoscale.

In one example, the polymer construct includes at least one extruded degradable polymer layer. The extruded degradable polymer layer can include a degradable host polymer material, which forms the degradable polymer matrix, and at least one star-shaped polymer loaded with one or more guest agents. The at least one star-shaped polymer loaded with one or more guest agents can be miscible with the host polymer material to provide a homogenous distribution of the star-shaped polymer and guest agent in the polymer layer.

In another example, a polymer construct includes a multilayer polymer composite sheet having coextruded, alternating first and second polymer layers. The first layers include a degradable host polymer material and at least one amphiphilic star-shaped polymer loaded with one or more guest agents. The at least one amphiphilic star-shaped polymer loaded with one or more guest agents can be miscible with the host polymer material. The second layers include a second polymer material immiscible with the host polymer material.

In another example, a method of forming a polymer construct includes extruding an amphiphilic star-shaped polymer loaded with a guest agent together with a degradable host polymer material to form at least one extruded polymer layer.

### BRIEF DESCRIPTION OF THE DRAWINGS

FIG. 1A is a schematic illustration of a coextrusion and layer multiplying device and process to form an example multilayer polymer composite film.

FIG. 1B is a schematic illustration of guest agents loaded onto star-shaped polymers so as to be encapsulated therein.

FIGS. 2A-2B illustrate a process for forming multilayer particles from the film of FIG. 1.

FIG. 3 illustrates an H-NMR spectrum of star-shaped copolymer PEI-b-PCL films (a) before purification and (b) after purification.

FIG. 4A illustrates a thermal stability study comprising TGA analysis of MO and PEI-b-PCL-MO.

FIG. 4B illustrates a comparative UV-Vis spectrum of MO and PEI-b-PCL-MO before and after heating up to 220° C.

FIG. 5 illustrates viscosity vs. temperature curves for different polymer melts determined by a melt flow indexer at a low flow rate.

FIG. 6 illustrates AFM phase images of (a) 50/50 PEO/PCL-PEI-b-PCL-MO and (b) 70/30 PEO/PCL-PEI-b-PCL-MO multilayer films.

FIG. 7 illustrates the X-ray diffraction spectrum of multilayer polymer composite films with different feed ratios.

FIG. 8 illustrates the infra-red spectrum of multilayer polymer composite films with different feed ratios.

FIG. 9 illustrates the release kinetics of different multilayer polymer composite films.

FIG. 10 illustrates the release kinetics of polymer nanosheets derived from multilayer polymer composite films including PEO/PCLPEI-b-PCL-MO with different pH values.

FIG. 11 illustrates the release kinetics of polymer nanosheets from multilayer polymer composite films including PEO/PCL-PEI-b-PCL-MO with and without a protecting layer in a phosphate buffered saline solution.

### DETAILED DESCRIPTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

The terms “biocompatible” and “biologically compatible” refer to materials that are, along with any metabolites or degradation products thereof, generally non-toxic to the recipient, and do not cause any significant adverse effects to the recipient, at concentrations resulting from the degradation of the administered materials. Generally speaking, biocompatible materials are materials that do not elicit a significant inflammatory or immune response when administered to a subject.

The term “biodegradable polymer” generally refers to a polymer that will degrade or erode by enzymatic action or hydrolysis under physiologic conditions to smaller units or chemical species that are capable of being metabolized, eliminated or excreted by the subject. The degradation time is a function of polymer composition, morphology, such as porosity, particle dimensions, and environment.

The term “guest agent” refers to a small organic or inorganic agent, such as a therapeutic agent or imaging agent that can be loaded within a star-shaped polymer described herein.

The term “controlled release” refers to control of the rate and/or quantity of a guest agent delivered using the polymer constructs described herein. The controlled release can be continuous or discontinuous, and/or linear or non-linear. This can be accomplished using one or more types of degradable polymer materials or compositions, drug loadings, inclusion of excipients or degradation enhancers, or other modifiers, administered alone, in combination or sequentially to produce the desired effect.

The term “effective amount” refers to an amount of guest agent that is sufficient to provide a desired effect. An effective amount in any individual case may be determined by one of ordinary skill in the art using routine experimentation.

The term “imaging agent” can refer to a biological or chemical moiety capable being loaded in the star-shaped polymers described herein and that may be used, for example, to detect, image, and/or monitor the presence and/or progression of a cell cycle, cell function/physiology, condition, pathological disorder and/or disease.

The term “subject” can be a human or non-human animal. Non-human animals include, for example, livestock and pets, such as ovine, bovine, porcine, canine, feline and murine mammals, as well as reptiles, birds and fish. Preferably, the subject is human.

This application describes polymer constructs and methods for fabricating extruded polymer constructs. The polymer constructs include an extruded degradable polymer matrix and amphiphilic star-shaped polymers loaded with one or more guest agents. The amphiphilic star-shaped polymers loaded with one or more guest agents can be uniformly dispersed and/or homogenously distributed within the polymer matrix and miscible therein. The polymer constructs can provide sustained and/or controlled release of the guest agent upon delivery and/or administration of the polymer construct to a site of interest.

Advantageously, the polymer construct can be formed using forced extrusion techniques and the guest agent upon release from the degradable polymer matrix can have the same or substantially similar structural (e.g., size, shape, and morphology), chemical, and/or biochemical (e.g., immune response) characteristics prior to extrusion. Moreover, loading the amphiphilic star-shaped polymer with the guest agent allows guest agents, which are not readily soluble in the polymer matrix by themselves, to be readily loaded and homogenously distributed and/or uniformly dispersed in the polymer matrix.

In some embodiments, where the polymer construct is used for therapeutic applications, the site of interest can be a cell or tissue of a subject. In other embodiments, where the polymer construct is used for agricultural applications, the site of interest can be a plant propagation material, a plant, part of a plant and/or plant organ.

In some embodiments, the polymer construct can be provided in a shape (e.g., a plurality of microparticles) that can be readily delivered to a subject to provide controlled and/or sustained release of the guest agent to cells and/or tissue of a subject. The polymer construct can be administered, injected, or implanted in a minimally invasive fashion in a subject in need thereof to treat diseases (e.g., cancer) and/or disorder in the subject. For example, the polymer constructs can take the form of films, sheets, particles, fibers, etc. on a microscale or nanoscale.

The star-shaped polymer includes a polymer core and a series of polymer branches extending from the core that form or define an outer shell around the core. The star-shaped polymer is amphiphilic such that the polymer core and polymer shell interact differently with water (e.g., a hydrophobic core can have hydrophilic branches and a hydrophilic core can have hydrophobic branches).

The star-shaped polymer can be formed from any amphiphilic compound, including amphiphilic dendrimers, hyberbranched polymers, and multiarm star polymers. Amphiphilic star polymers comprising branched polymer arms have been disclosed. Qiao, et al., WO2007/051252 A1, discloses biodegradable star polymers formed by ring opening polymerization of cyclic carbonyl monomers using metal catalysts. Meier et al., WO2007/048423 A1; Lin et al., Biomolecules, vol. 9(10), (2008), pages 2629-36; and An et al., Polymer, vol. 47, (2006), pages 4154-62 describe organic soluble (i.e., non-water soluble) amphiphilic core-shell star polymers. The “shell” portion contains the polymer arms, generally attached to a static core (e.g., dendrimers, pentaerythritol, and the like).

The shell has an inner hydrophilic region and an outer hydrophobic region, each of various compositions. Alternatively, Kreutzer et al., Macromolecules, vol. 39(13), (2006), pages 4507-16, and Zhao et al., U.S. Pat. No. 7,265,186 B2, constructed water soluble amphiphilic core-shell star polymers comprising arms that present a hydrophilic outer region of various compositions and a hydrophobic inner region of various compositions, attached to a static core (e.g., dendrimers, pentaerythritol, etc). Fukukawa, et al., Biomacromolecules, vol. 9(4), (2008), pages 1329-39, disclose water soluble star polymers comprising hydrophilic outer and inner shell regions, attached to a microgel core of varying hydrophobicity composed of either poly(ethylene glycol diacrylate) or poly(divinylbenzene). Conversely, Gao, et al., Macromolecules, vol. 41(4), (2008), pages 1118-1125 describe star polymers comprising a hydrophobic outer shell and a hydrophobic inner shell attached to a microgel core.

In some embodiments, the star-shaped polymer can include a core-shell type amphiliphilic block copolymer that has a polyethyleneimine (PEI) core and a plurality of poly(caprolactone) (PCL) branches. The PEI core can have a number average molecular weight of, for example, about 1,000 g/mol to about 25,000 g/mol. The degree of polymerization of the caprolactone can be, for example, about 10 to about 50. The number of PCL branches attached to the PEI core can be about 30 to about 300.

In some embodiments, the star-shaped polymer can be modified with, for example, radical-crosslinkable methacrylate groups or polyethylene glycol such that the shell has both inner and outer components around the core (not shown). The star-shaped polymer can be configured to encapsulate apolar or polar guest agents, depending on the polarity (or lack thereof) of the core and shell.

The guest agent can be hydrophobic or hydrophilic and have a similar polarity (e.g., polar or apolar) as the polarity of the core to allow the guest agent to be readily loaded within the core and released in a controlled manner when the polymer construct is exposed to a particular medium, such as a solvent, air, water, etc. The guest agent release properties/conditions can be adjusted by modifying the polymer construct thickness, the degradable polymer matrix, and/or the intensity of the connection between the star-shaped polymer and the guest agent.

The guest agent can include any compound or material that can be readily loaded on or within the star-shaped polymer core by, for example, a liquid-liquid phase transfer method. The guest agent can include biologically active substances. Examples of biologically active substances include biomolecules (e.g., DNA, genes, peptides, proteins, enzymes, lipids, phospholipids, and nucleotides), natural or synthetic organic compounds (e.g., drugs, dyes, synthetic polymers, oligomers, and amino acids), inorganic materials (e.g., metals and metal oxides), chromophores that aid in diagnostics (e.g., porphyrinoid compounds, including porphyrins and phthalocyanines), radioactive variants of the foregoing, and combinations of the foregoing. Some of the biologically active substances can alter the chemical structure and/or activity of a cell, or can selectively alter the chemical structure and/or activity of a cell type relative to another cell type.

As an example, one desirable change in a chemical structure can be the incorporation of a gene into the DNA of the cell. A desirable change in activity can be the expression of the transfected gene. Another change in cell activity can be the induced production of a desired hormone or enzyme. A desirable change in cell activity can also be the selective death of one cell type over another cell type. No limitation is placed on the relative change in cellular activity caused by the biologically active substance, provided the change is desirable and useful. Other biologically active materials herein improve diagnostic capability without necessarily altering the structure or activity of the tissue, organ, bone, or cell. These include image contrast enhancing agents for magnetic resonance imaging and x-ray imaging. To this end, the guest agent can comprise a metal, including one or more of the above-described restricted metals.

In some embodiments, the guest agent can include an imaging agent. Examples of imaging agents include fluorescent or non-fluorescent compounds or dyes (e.g., methyl orange, conge red, Thio-michler's ketone, ethidium bromide or methylene blue), radioactive isotopes, and MRI contrast agents. For example, in some embodiments, the imaging agent is a fluorescent molecule for fluorescent imaging. The imaging agent can be any material having a detectable physical or chemical property. Such imaging agents have been well-developed in the field of fluorescent imaging, magnetic resonance imaging, positive emission tomography, or immunoassays and, in general, most any imaging agent useful in such methods can be applied to the present invention. Thus, an imaging agent can be any composition detectable by spectroscopic, photochemical, biochemical, immunochemical, electrical, optical or chemical means.

Means of detecting imaging agents are well known to those of skill in the art. Thus, for example, where the imaging agent is a radioactive compound, means for detection include a scintillation counter or photographic film as in autoradiography. Where the imaging agent includes a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally, simple colorimetric labels may be detected simply by observing the color associated with the label.

In other embodiments, the guest agent can be a therapeutic agent. Specific non-limiting examples of therapeutic agents include analgesics and anti-inflammatory agents, anthelmintics, anti-arrhythmic agents, anti-asthma agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents and immunosuppressants, anti-protozoal agents, anti-thyroid agents, anti-tussives, anxiolytic, sedatives, hypnotics and neuroleptics, β-blockers, cardiac inotropic agents, diuretics, anti-parkinsonian agents, gastrointestinal agents, histamine H,-receptor antagonists, keratolytics, lipid regulating agents, muscle relaxants, anti-anginal agents, nutritional agents, analgesics, sex hormones, stimulants, cytokines, peptidomimetics, peptides, proteins, toxoids, antibodies, nucleosides, nucleotides, genetic material, and nucleic acids. suitable agents include water soluble complex polysaccharides having at least two and preferably three or more monosaccharide units and additionally containing one or more of the following chemical substituents: amino groups (free or acylated), carboxyl groups (free or acylated), phosphate groups (free or esterified) or sulfate groups (free or esterified).

Preferred water soluble active agents include RGD fibrinogen receptor antagonists, enkephalins, growth hormone releasing peptides and analogues, vasopressins, desmopressin, luteinizing hormone releasing hormones, melanocyte stimulating hormones and analogues, calcitonins, parathyroid hormone, PTH-related peptides, insulins, atrial natriuretic peptides and analogues, growth hormones, interferons, lymphokines, erythropoietins, interleukins, colony stimulating factors, tissue plasminogen activators, tumor necrosis factors, complex polysaccharides, and nucleosides, nucleotides and their polymers

In some embodiments, the therapeutic agents used as guest agents are small molecule antitumor agents. Examples of small molecule antitumor agents include angiogenesis inhibitors such as angiostatin K1-3, DL-α-difluoromethyl-ornithine, endostatin, fumagillin, genistein, minocycline, staurosporine, and thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine β-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, deguelin, 5,6-dichlorobenz-imidazole 1-beta-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-.alpha., rapamycin, thapsigargin, and bikunin, and derivatives thereof.

The star-shaped polymer loaded with the guest agent can be extruded with one or more degradable host polymers to form at least one layer of the polymer construct. The star-shaped polymer loaded with the guest agent can be mixed or loaded with the degradable host polymer material at loading levels of about 1%, 5%, 20%, 25% or more to provide a polymer layer with star-shaped polymer and guest agent loading levels of about 1%, 5%, 20%, 25% or more. The loading level can influence the release profile from the degradable polymer matrix of the polymer construct. In some embodiments, increasing the loading level can increase the amount of guest agents initially released from the polymer construct.

The degradable host polymer can include or be made of a melt processable degradable polymer material. The melt processable degradable polymer material can be, for example, hydrolytically degradable, biodegradable, thermally degradable, and/or photolytically degradable.

The degradable polymer material can also have a melt temperature that allows the degradable polymer to be readily processed by, for example, melt extrusion, and below the degradation temperature of the star-shaped polymer and guest agent. For example, the degradable polymer material can have a melt temperature below about 120° C., about 110° C., about 100° C., about 90° C., about 80° C., or about 70° C. and be readily extruded without the aid of solvents with the star-shaped polymer and guest agent to form the extruded polymer layer.

Melt processable degradable polymers can include, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, biocompatible, biodegradable, or bioerodible polymers include poly(lactic acid) (PLA), poly(glycolic acid) (PGA), poly(lactic acid-co-glycolic acid)s (PLGAs), polyanhydrides, polyorthoesters, polyetheresters, PCLs, polyesteramides, poly(butyric acid), poly(valeric acid), polyvinylpyrrolidone (PVP), polyvinyl alcohol (PVA), polyethylene glycol (PEG), block copolymers of PEG-PLA, PEG-PLA-PEG, PLA-PEG-PLA, PEG-PLGA, PEG-PLGA-PEG, PLGA-PEG-PLGA, PEG-PCL, PEG-PCL-PEG, PCL-PEG-PCL, copolymers of ethylene glycol-propylene glycol-ethylene glycol (PEG-PPG-PEG, trade name of Pluronic or Poloxamer) and copolymers and blends of these polymers.

The degradable polymers described herein can have a variety of molecular weights. The polymers may, for example, have molecular weights of at least about 5 kD, at least about 10 kD, at least about 20 kD, at least about 22 kD, at least about 30 kD, or at least about 50 kD.

The degradable polymers and derivatives thereof can be selected such that the star-shaped polymer is readily miscible in the degradable polymers during extrusion. Such degradable polymers forming the host polymer can be formed of the same or substantially similar monomers as the monomers used to form the outer shell of the star-shaped polymers. Advantageously, the degradable polymer can be adapted to have a desired degradation rate. Alternatively or additionally, the degradation rate may be fine-tuned by associating or mixing other materials (e.g., non-degradable materials) with one or more degradable polymer materials.

In general, a degradation rate as used herein can be dictated by the time in which a material degrades a certain percentage (e.g., 50%) in a certain condition (e.g., in physiological conditions). In some embodiments, the degradation time of the degradable polymer of the polymer construct or a portion of the polymer construct as described herein can have a wide range. In some embodiments, the degradation time may be greater than 1 minute, 5 minutes, 30 minutes, 1 hour, 2 hours, 5 hours, 12 hours, 24 hours, 1.5 days, 2 days, 5 days, 7 days, 15 days, 30 days, 2 months, 6 months, 1 year, 2 years, or even 5 years. In some embodiments, the degradation time may be about or less than 10 years, 5 years, 2 years, 1 year, 6 months, 2 months, 30 days, 15 days, 7 days, 5 days, 2 days, 1.5 days, 24 hours, 12 hours, 5 hours, 2 hours, 1 hour, 30 minutes or even 5 minutes. The degradation time may be in a range of 12-24 hours, 1-6 months, or 1-5 years. In some embodiments, the degradation time may be in a range of any two values above.

In other embodiments, the degradable polymer of the polymer construct is designed to release star-shaped polymer and/or guest agent over a period of days to weeks. Factors that affect the duration of release include pH of the surrounding medium (e.g., higher rate of release at pH 5 and below due to acid catalyzed hydrolysis of the polymer) and polymer composition. Aliphatic polyesters differ in hydrophobicity and that, in turn, affects the degradation rate. The degradation rate of these polymers, and often the corresponding drug release rate, can vary from days to months and easily varied.

The extruded polymer layer can also be coextruded with another polymer material, divided, and stacked in a repeating, alternating manner to form a multilayer polymer composite construct. The multilayer construct can be immersed in a solvent and/or mechanically cut or chopped into polymer composite particles or fibers.

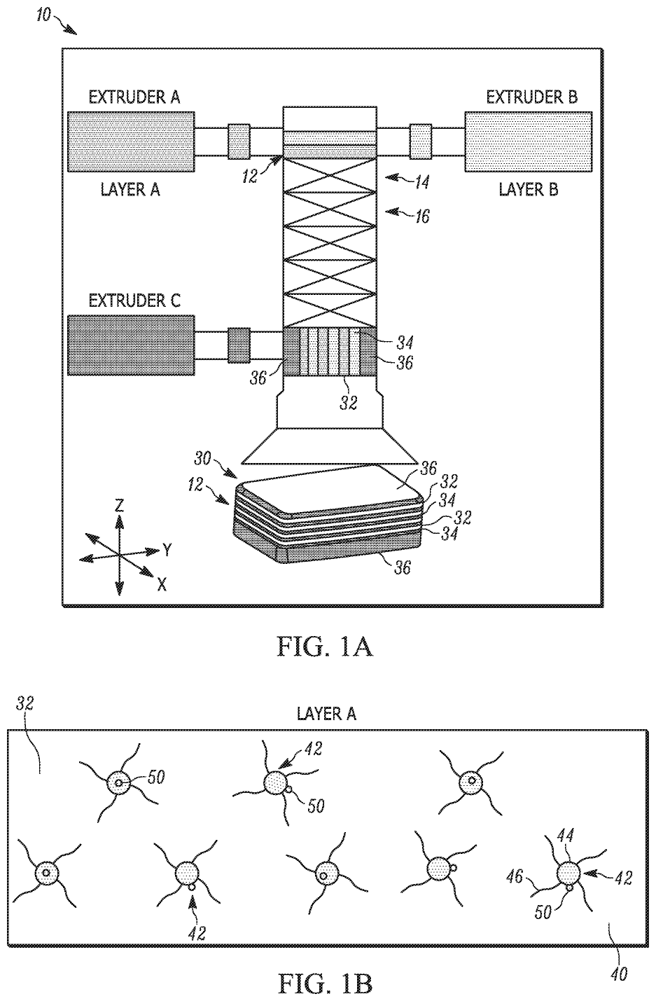

FIG. 1A illustrates an example coextrusion and multiplying or multilayering process 10 used to form a polymer construct—in this case a multilayer polymer composite film or sheet 30. In the process 10, a first polymer layer 32 and a second polymer layer 34 are provided. The first layer 32 is formed from a first polymer material (A). The second polymer layer 34 is formed from a second polymer material (B). The second polymer material (B) has a substantially similar viscosity to the first polymer material (A) and is substantially immiscible with the first polymer material (A) when coextruded.

The first and second polymer materials (A), (B) are coextruded to form a polymer composite having a plurality of discrete layers 32, 34 that collectively define a polymer composite stream 12. It will be appreciated that one or more additional layers formed from the polymer materials (A) or (B) or formed from different polymer materials may be provided to produce a polymer composite stream 12 that has at least three, four, five, six, or more layers of different polymer materials. Although one of each layer 32 and 34 is illustrated in the composite stream 12 of FIG. 1A it will be appreciated that the polymer composite stream 12 may include, for example, up to thousands of each layer 32, 34. In any case, the polymer composite stream 12 is then divided, stacked, and multiplied to form the multilayer polymer composite film 30 having, for example, hundreds or thousands of layers 32, 34.

One or more dies—two of which are indicated at 14 and 16 in FIG. 1A—are used to multiply the coextruded layers 32, 34. Each layer 32, 34 in the completed multilayer polymer composite film 30 extends within an x-y plane of an x-y-z coordinate system. Each layer 32, 34 initially extends in the y-direction. The y-direction defines the length of the layers 32, 34 and extends in the general direction of material flow through the dies 14, 16. The x-direction extends transverse (e.g., perpendicular) to the y-direction and defines the width of the layers 32, 34. The z-direction extends transverse (e.g., perpendicular) to both the x-direction and the y-direction and defines the height or thickness of the layers 32, 34.

Once the multilayer film 30 is formed a detachable skin or surface layer 36 can be applied to the top and bottom of the film via coextrusion prior to the film exiting the last die. The skin layers 36 can be applied such that the film 30 is sandwiched therebetween. The skin layer 36 may be formed from the first polymer material (A), the second polymer material (B) or a third polymer material (C) different from the first and second polymer materials (A), (B). One or both of the skin layers 36 can, however, be omitted (not shown).

In one example, the first polymer material (A) includes a degradable host polymer 40 and at least one amphiphilic star-shaped polymer 42 dispersed therein. The star-shaped polymer 42 includes a core 44 and branches 46 extending outwardly from the core. The branches 46 form a shell extending around the core 44 and can number, for example, between a few branches and hundreds of branches. A guest agent 50 is loaded on and/or within the core 44.

The guest agent 50 is integrated into the host polymer 40 of the first polymer material (A) prior to coextruding the first and second polymer materials (A), (B). In one example, solutions of the guest agent 50 and the star-shaped polymer 42 are mixed together. In one instance, the guest agent 50 solution is an aqueous solution and the host polymer 40 solution is a chloroform solution. The guest agent 50 and host polymer 40 solutions can be combined in the same or different volumes with one another.

It will be appreciated that one type of star-shaped polymer could be configured to encapsulate multiple, different types of guest agents 50. Moreover, the host material 40 could be configured to accommodate more than one star-shaped polymer 42.

In any case, subsequent separating, drying, and solvent removal from the combined solution mixture produces a polymer solid that contains the guest agent 50 loaded with and/or encapsulated within the core 44 of the star-shaped polymer 42. When the guest agent 50 is mixed with the star-shaped polymer 42, the guest passes through the shell to the core where it becomes coupled thereto by, for example, electrostatic interaction. The coupling may be internal or external to the core 44 (both shown in FIG. 1B) but in either case the guest agent 50 becomes encapsulated inside the star-shaped polymer 42. The core 44 therefore supplies a confined microenvironment for the accommodation of guest agents 50 with similar polarity 46. It has been known that enlarging the size of the core 44 or the polarity difference between the core 44 and the shell 46 can enhance the star-shaped polymer 42 guest agent 50 encapsulation capability.

This solid can then be extruded with one or more degradable host polymer materials 40 to form the first polymer material (A). The shell 46, having a different polarity from the core 44 and the guest agent 50, helps stabilize the interface between the core and the host polymer material 40.

The core 44 and the shell 46 of the amphiphilic star-shaped polymer 42 are selected to be miscible with the host polymer 40. As a result, the star-shaped polymer 42 is homogenously distributed within the host polymer 40 when the two are extruded to form the first layer 32.

The second polymer material (B) forms the second polymer layer 34 and is coextruded with the first polymer material (A). The second polymer material (B) can include any polymer that can be readily extruded with the first polymer material (A) (e.g., has a substantially similar viscosity during extrusion) and is substantially immiscible with the first polymer material (A) to provide a separate polymer layer upon coextrusion. Examples of polymers that can be used as the second polymer material (B) include polyethers, such as polyethylene oxide (PEO); polyesters, such as poly(ethylene terephthalate) (PET), poly(butylene terephthalate), PCL, and poly(ethylene naphthalate)polyethylene; naphthalate and isomers thereof, such as 2,6-, 1,4-, 1,5-, 2,7-, and 2,3-polyethylene naphthalate; polyalkylene terephthalates, such as polyethylene terephthalate, polybutylene terephthalate, and poly-1,4-cyclohexanedimethylene terephthalate; polyimides, such as polyacrylic imides; polyetherimides; styrenic polymers, such as polystyrene (PS), atactic, isotactic and syndiotactic polystyrene, α-methyl-polystyrene, para-methyl-polystyrene; polycarbonates, such as bisphenol-A-polycarbonate (PC); polyethylenes oxides; poly(meth)acrylates such as poly(isobutyl methacrylate), poly(propyl methacrylate), poly(ethyl methacrylate), poly(methyl methacrylate), poly(butyl acrylate) and poly(methyl acrylate) (the term “(meth)acrylate” is used herein to denote acrylate or methacrylate); cellulose derivatives; such as ethyl cellulose, cellulose acetate, cellulose propionate, cellulose acetate butyrate, and cellulose nitrate; polyalkylene polymers such as polypropylene, polyethylene, high density polyethyelene (HDPE), low density polyethylene (LDPE), polybutylene, polyisobutylene, and poly(4-methyl)pentene; fluorinated polymers such as perfluoroalkoxy resins, polytetrafluoroethylene, fluorinated ethylene-propylene copolymers, polyvinylidene fluoride, polyvinylidene difluoride (PVDF), and polychlorotrifluoroethylene and copolymers thereof; chlorinated polymers such as polydichlorostyrene, polyvinylidene chloride and polyvinylchloride; polysulfones; polyethersulfones; polyacrylonitrile; polyamides such as nylon, nylon 6,6, polycaprolactam, and polyamide 6 (PA6); polyvinylacetate; polyether-amides.

Copolymers, such as styrene-acrylonitrile copolymer (SAN), preferably containing between 10 and 50 wt %, preferably between 20 and 40 wt %, acrylonitrile, styrene-ethylene copolymer; and poly(ethylene-1,4-cyclohex-ylenedimethylene terephthalate) (PETG), can also be used as either the host polymer material 40 in the first polymer material (A) or the second polymer material (B). Additional polymer materials include an acrylic rubber; isoprene (IR); isobutylene-isoprene (IIR); butadiene rubber (BR); butadiene-styrene-vinyl pyridine (PSBR); butyl rubber; chloroprene (CR); epichlorohydrin rubber; ethylene-propylene (EPM); ethylene-propylene-diene (EPDM); nitrile-butadiene (NBR); polyisoprene; silicon rubber; styrene-butadiene (SBR); and urethane rubber. Polymer materials can also include block or graft copolymers.

In addition, each individual layer 32, 34 may include blends of two or more of the above-described polymers or copolymers. The components of the blend can be substantially miscible with one another yet still maintain substantial immiscibility between the layers 32, 34.

In some embodiments, the first and second polymer materials (A), (B) comprising the layers 32, 34 can include organic or inorganic materials, including nanoparticulate materials, designed, for example, to modify the mechanical properties of the polymer materials (e.g., tensile strength, toughness, and yield strength)\. It will be appreciated that potentially any extrudable polymer material can be used as either the host polymer 40 in the first polymer material (A) or the second polymer material (B) so long as upon coextrusion such polymer materials (A), (B) are substantially immiscible, have a substantially similar viscosity, and form discrete layers or polymer regions.

The star-shaped polymer 42 can be formed from any amphiphilic compound. The star-shaped polymer 42 can be configured to encapsulate apolar or polar guest agents 50, depending on the polarity of the core 44. Advantageously, during coextrusion the star-shaped polymer 42 limits diffusion of the guest agents 50 from the first polymer layer 32 to the second polymer layer 34.

In one example, the host polymer 40 is PCL, the star-shaped polymer 42 is PEI-b-PCL, and the guest agent 50 is methyl orange (MO) and, thus, the first polymer material (A) is PCL-PEI-b-PCL-MO. The second polymer material (B) is PEO.

Referring to FIG. 1, the multilayer film 30 can be maintained as a sheet. The sheet form of the multilayer film 30 can retain both layers 32, 34 (as shown). Alternatively, a solvent can be used to remove the one or more of the second layers 34. In one example, the sheet includes only a single layer 32 and none of the layers 34 or the skin layer 36 (not shown).

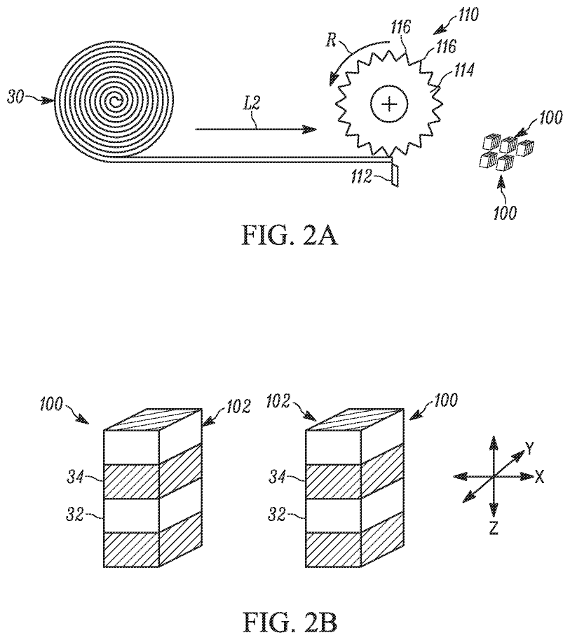

Alternatively, as shown in FIGS. 2A-2B, the multilayer film 30 can be mechanically chopped and/or cut into multilayer polymer composite particles 100. In this instance, the multilayer film 30 can be provided in a rolled form and fed in the manner L to a machine 110 that includes a stationary blade 112 and blade 114 that rotates in the manner R. The blades 112, 114 cooperate to cut or chop the multilayer polymer composite film 30 into particles 100 having a round or polygonal shape, depending on the shapes of the blades 112, 114. The circumferential spacing between the cutting tines 116 on the blade 114 help to determine the dimensions of the particles 100.

The size of the particles 100 depends on the thickness in the z-direction of the multilayer polymer composite film 30 when the coextrusion process 10 is complete. That said, the particles 100 can be formed as nanoparticles or microparticles. In another example, the multilayer film 30 can be formed into elongated, square or rectangular fibers in a manner similar to the production of the particles 100 (i.e., by cutting or chopping the multilayer film). The size of the particles 100 can be, for example, about 1 μm, about 10 μm, about 25 μm, about 50 μm, about 100 μm or more. In some embodiments, the size of the particles 100 can be about 1 μm to about 100 μm, or about 10 μm to about 25 μm.

The encapsulation system described herein can be used in a variety of applications due to the ability of the amphiphilic star-shaped polymer 42 within the polymer material (A) to receive a range of guests agents 50 to meet the intended application. The encapsulation system can, for example, be used in the food industry (e.g., encapsulation of flavor and other food additives); the oil and gas industry (e.g., encapsulation of corrosion inhibitors); agriculture (e.g., encapsulation of fertilizers and pesticides); personal care applications (e.g., encapsulation of vitamin C, insect repellant and lotions); catalysis (e.g., encapsulation of a catalyst); reaction vessels; and pharmaceutical applications (e.g., encapsulation of bio-active molecules such as cancer drugs or other controlled release or drug delivery technology).

In some embodiments, the polymer construct can used to provide sustained and/or controlled delivery of guest agent to a target tissue in a subject. The polymer construct can be in situ delivered and/or administered to the tissue of the subject. Upon delivery and/or administration of the polymer construct to tissue, the polymer construct can degrade and/or erode by, for example, hydrolysis to release the guest agent to the tissue.

When used in vivo, the polymer constructs can be administered as a pharmaceutical composition and a pharmaceutically acceptable carrier. The polymers constructs, or pharmaceutical compositions comprising these constructs, may be administered by any method designed to provide the desired effect. Administration may occur enterally or parenterally; for example orally, rectally, intracisternally, intravaginally, intraperitoneally or locally. Parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, intracranial and intrathecal administration for CNS tumors, and direct application to the target area, for example by a catheter or other placement device.

One skilled in the art can readily determine an effective amount of the polymer constructs to be administered to a given subject, by taking into account factors such as the size and weight of the subject; the extent of disease penetration; the age, health and sex of the subject; the route of administration; and whether the administration is local or systemic. Those skilled in the art may derive appropriate dosages and schedules of administration to suit the specific circumstances and needs of the subject. For example, where the guest agent is an anti-cancer agent suitable doses of the guest agent to be administered can be estimated from the volume of cancer cells to be killed or volume of tumor to which the guest agent is being administered.

Advantageously, where the guest agent is a therapeutic agent, the polymer construct can provide a slow-release and/sustained formulation of the guest agent that maintains sustained administration without the need for repeat injections. The release of the guest agent can be constant and sustained for about 1 day, 2 days, 3 days, 4 days, 5 days, 6 days, 1 week, 2 weeks or more. The constant release can be sustained between subsequent administrations. The release of the guest agent from the degradable polymer matrix can be at least partially defined by the swelling and degradation rate of the degradable polymer material under physiological conditions.

The following example has been included to more clearly describe particular embodiments described herein. However, there are a wide variety of other embodiments within the scope of the present invention, which should not be limited to the particular examples provided herein.

### Example

In this Example, we investigated the controlled release property of polymer nanosheets, which was generated from coextruded multilayer polymer composite films having alternating layers ABAB. In layer A, a star-shaped polyethylenimine-block-poly(ε-caprolactone) (PEI-b-PCL) encapsulating a hydrophilic guest (HG) was melt blended with linear PCL. Layer B was formed from water soluble PEO. The forced assembly multilayer coextrusion of layer A and layer B generated a continuous ABAB-type multilayer film. The multilayer film was immersed in a phosphate buffered saline (PBS) solution to study the release kinetics of the HG from the layer A.

### Chemicals and Materials

PCL with a reported molecular weight of 120 kg/mol (Capa 6800) and PEO with a molecular weight of 200 kg/mol (PolyOx WSR N-80) and 100 kg/mol (PolyOx WSR N-10) were obtained from the Dow Chemical Company. Hyperbranched polyethylenimines (PEI10K) from Alfa Aesar were dried under vacuum prior to use. ε-Capralatone (CL, 99%, Alfa Aesar) was distilled from CaH₂ under reduced pressure. Tin(II) 2-ethylhexanoate [Sn(Oct)2, 97%] and methyl orange (MO) were purchased from Alfa Aesar and used directly.

### Instrumentation

Thermal transitions of polymer films were obtained using DSC (TA instruments Q100) at a heating rate of 10° C./min over a temperature range of about −80 to 200° C. The thermal properties of the polymers were measured by thermogravimetric analysis (TGA) on a TA Instruments, TGA 2920. The samples were heated up to 220° C. at a heating rate of 15° C./min under a dry nitrogen atmosphere (flow rate: 70 mL/min) on a TA Instruments 2950 thermogravimetric analyzer. X-ray diffraction (XRD) measurements were conducted via a Rigaku diffractometer in transmission mode with Cu Kα X-rays (λ=0.154 nm) operating at 40 kV and 40 mA. The experiment was carried out at 25° C. with a scan speed of 0.5°/min over a scan range of 5° to 40°.

Atomic force microscopy-phase imaging (AFM, Park system NX 10) was used to visualize the layered structure of the multilayer film. The embedding process and the preparation of microtomed cross-sections were accomplished using known methods. UV-vis spectra were recorded on a 5 Agilent 8453 spectrometer and fluorescence spectra were obtained using Perkin-Elmer LS45 luminescence spectrometer. Fourier transform infrared spectroscopy (FTIR) measurement was performed using a Digilab FTS 7000 step scan spectrometer. FTIR images were taken using the Digilab Stinggray imaging system consisting of the Digilab FTS 7000 spectrometer and a 32×32 mercury-cadmium-telluride IR imaging focal plane array (MCT-FPA) image detector with average spatial area of 176 μm×176 μm in reflectance mode. 128 scans were performed in obtaining the images, which were processed using the Win-IR Pro 3.4 software package.

### Dye Loading

An aqueous solution of MO (1.5 mg/mL) and chloroform solution of PEI-b-PCL (10 mg/mL) were separately prepared. An equivalent volume of the two solutions was mixed together and shaken for 5 minutes. The organic layer was separated from the mixture and dried by sodium sulfate. The solvent was then removed to produce a polymer solid with MO encapsulated inside, namely, PEI-b-PCL-MO. Layer A was prepared by extruding 10% PEI-b-PCL-MO and 90% PCL via twin-screw extruder to form PCL-PEI-b-PCL-MO.

### Layer-Multiplying Co-Extrusion

Both layer A (PCL-PEI-b-PCL-MO) and layer B (PEO) were dried under vacuum before processing. A multilayer film having 256 alternating layers of PEO and PCL-PEI-b-PCL-MO was fabricated using the layer multiplication process shown in FIG. 1. To ensure matching viscosities of the two polymer melts, the extruder, multiplier elements and die temperatures were all set to 200° C. A PE outer protecting layer was coextruded over the top and bottom of the multilayer film. The thickness of the PCL-PEI-b-PCL-MO layer was varied by adjusting the feed ratio of each polymer material to the extruder. In one example, the 70/30 and 50/50 ratio PEO/PCL-PEI-b-PCL-MO multilayer films had thicknesses of about 52 and about 85 nm, respectively.

### Polymer Nanosheet for Release Study

PBS solutions at a concentration of 0.1 M/L but at different pH values were prepared. Three pieces of the multilayer film having dimensions of about 5 cm long and about 0.5 cm wide were immersed in equivalent volumes of PBS solutions at 37° C. At predetermined time intervals, 2 mL of aqueous solution was collected for a UV-Vis (ultraviolet-visible) absorption measurement. At the same time, 2 mL of fresh PBS solution was refilled to the vial.

### Initial Release Rate Calculation and Simulation

Data were expressed as mean±SD. The experimental data was fitted against a trend line having the formula y=a−bc^{t}. The initial release rate of the MO hydrophilic agent was calculated by obtaining the derivative

$$( i . e . , \frac{dy}{dt} = - b log ( c ) )$$

of the formula. A simulation was performed using the Transport of Dilute Species Model Comsol Multiphysics 4.3 software.

### Layer Multiplying Co-Extrusion

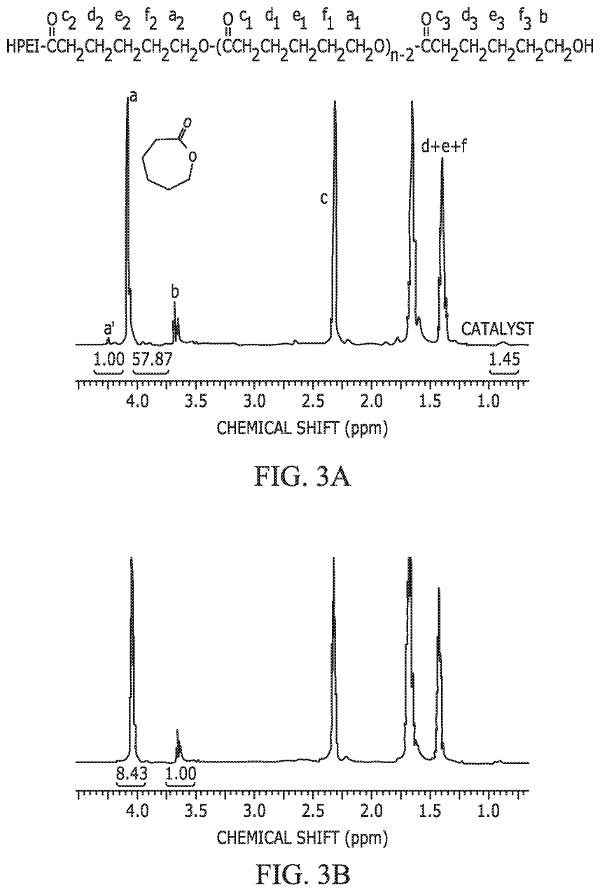

MO was employed as the anionic hydrophilic guest because its release kinetics could be easily monitored by the UV-Vis absorption spectroscopy. The star-shaped copolymer PEI-b-PCL with hydrophilic PEI as the core and hydrophobic PCL as the shell was well reported to encapsulate anionic guests (e.g., methyl orange and conge red) inside the hydrophilic core. Synthesis of star-shaped copolymer PEI-b-PCL was conducted as previously discussed. The chemical structure and H-NMR spectrum of PEI-b-PCL is shown in FIG. 3. The insertion of PEI-b-PCL not only provided the physical affinity between the host polymer and the hydrophilic guest, but also allowed for a homogeneous host polymer-guest agent mixture rather than a direct mixture of PCL and MO.

The encapsulation of MO by PEI-b-PCL was conducted by a liquid-liquid phase transfer method. A polymer solid (PEI-b-PCL-MO) was mixed with commercially available, linear PCL. The polymer mixture was extruded by a twin-screw extruder to generate homogeneous polymer granules or particles (a1) (layer A). As a control study, a mixture of MO and linear PCL was also extruded in the same manner to generate polymer granules or particles a2.

Due to the high polarity and melting point of MO, it was difficult to extrude the MO with PCL polymer melt and, thus, a large amount of MO was left in the setup. As a result, the color of the polymer granules (a2) was lighter than the PEI-b-PCL granules (a1), even with a higher MO feed ratio (1.5% compared to 1.0% by weight ratio).

To compare the dye distributions, two polymer films were fabricated by melt compression from these two polymer granules (a1), (a2). FTIR image technique was employed to monitor the dye distribution in micro-scale. In the FTIR spectrum of the multilayer film films, the peaks at 1607 cm⁻¹ and 1725 cm⁻¹ were assigned to the aromatic carbon-carbon stretching of MO and to the C═O stretching of PCL. The images focused at 1725 cm⁻¹ revealed a wide PCL distribution for both multilayer films.

The images at 1607 cm⁻¹ corresponding to the MO exhibited very different PCL distribution behaviors. More specifically, the multilayer film with the star-shaped copolymer had homogeneous dye distribution, whereas the one without the star-shaped copolymer had a remarkable inclination of dye aggregation. Microscopic images of the multilayer film from the granules (a1) also showed different color intensity in different areas, while the multilayer film from the granules (a2) displayed similar colors with different areas. The star-shaped copolymer also functioned as a stable guest-host, which was particularly effective in limiting diffusion during the further layer multiplication coextrusion process.

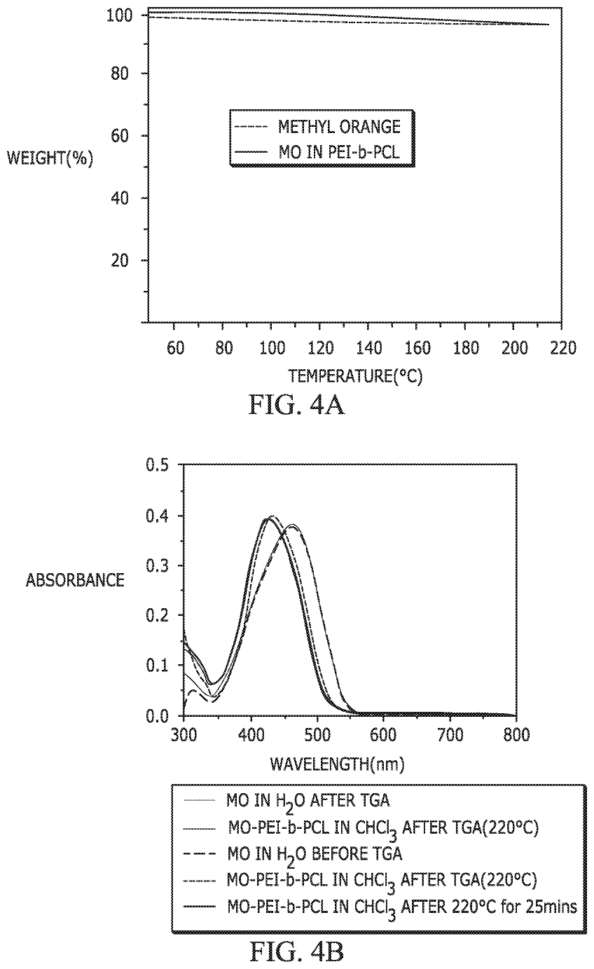

The polymer granule (a1) was immersed in water solution and shaken for 5 minutes. The solution was taken for a UV-Vis measurement (see FIG. 4(b)). A high intensity adsorption peak corresponding to the MO revealed large amount of diffusion. On the other hand, the solution with the star-shaped copolymer showed very low UV-Vis absorption.

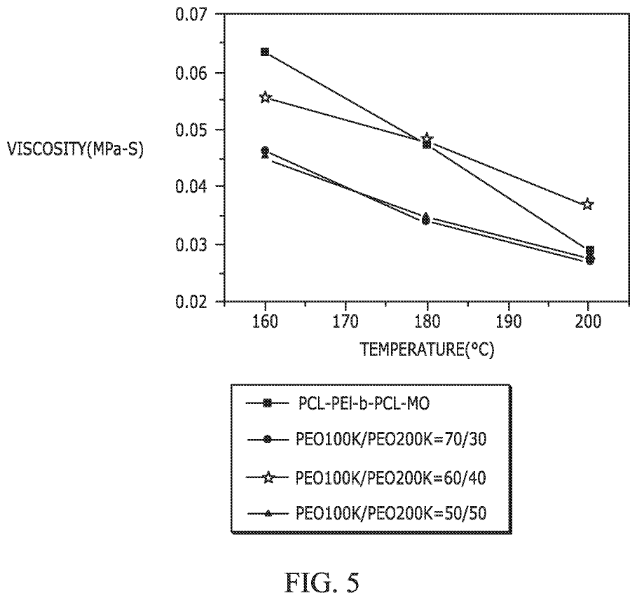

The thermal property of the polymer-guest system was also investigated by the TGA to ensure system stability during the high temperature layer multiplying coextrusion process. Referring to FIG. 4(a), the polymer-guest system PEI-b-PCL-MO showed no weight loss up to 220° C. The conjugation structure of MO remained intact according to comparative UV-Vis measurements. A viscosity-match temperature of layer multiplying co-extrusion was determined by a melt flow indexer (MFI) at a low shear rate as shown in FIG. 5. A 60/40 feed ratio of PEO100K/PEO200K in layer B was obtained with the processing temperature of 200° C. selected based on material rheological compatibility. The ABAB-type layer multiplication process was conducted accordingly with PE as the protective surface layer and the technique described herein used to form the multilayer film.

### Characterization of Multilayer Polymer Film

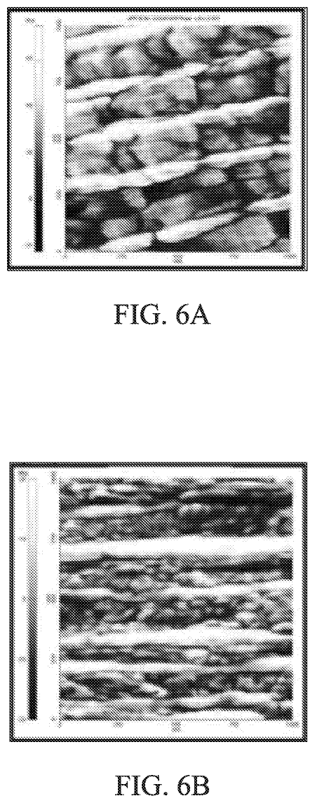

As shown in FIG. 6(a), the multilayer nanostructure was first analyzed by utilizing phase contrast atomic force microscopy (AFM). The layer thickness varied approximately ±18% of the nominal value due to the relatively thinner polymer layers (<100 nm for the PCL-PEI-b-PCL-MO layer). The difference in elasticity between the two coextruded polymers and the polymer layers with nano-scaled thickness remained integral. The PCL layer thicknesses were determined from AFM images to be 85±13 nm and 52±12 nm for PEO/PCL-PEI-b-PCL-MO=50/50 (FIG. 6(a)) and PEO/PCL-PEI-b-PCL-MO=70/30 (FIG. 6(b)), respectively.

With amorphous substrates, such as PS and PMMA, the crystallization habit of crystalline polymers can be well controlled in some degree. PCL is a semi-crystalline polymer with crystallinity of around 45%, while PEO shows higher crystallinity (around 72%). Several sharp peaks in

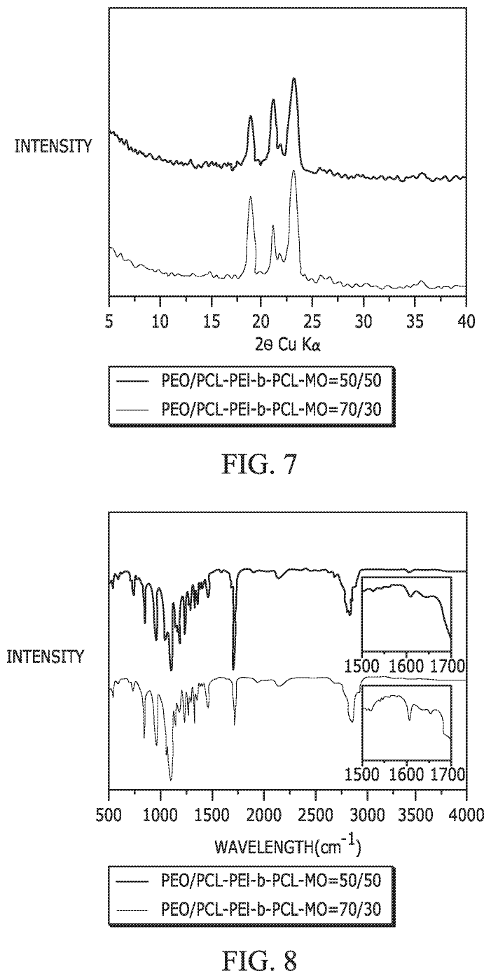

X-ray diffraction spectrum (FIG. 7) confirmed the crystalline structure of the multilayer film. The typical peaks at 2θ=19.1° and 23.4° correspond with the crystalline PEO. A scattering angle 2θ=21.3° (i.e., reflections from the PCL (110) planes) could be clearly observed, while the scattering angle from PCL (200) reflections (2θ=23.5°) was overlapped by the PEO crystalline peaks.

Differential scanning calorimetry (DSC) was also employed to measure the crystallinity of the multilayer film. The DSC curves for both multilayer films showed two peaks in both heating and cooling runs, which correspond with the melting temperature and crystallization temperature, respectively. The heating thermogram showed a PCL melting endotherm at 55° C. (T_{m,PCL}), while PEO melted at a higher temperature of 63° C. (T_{m,PEO}). Moreover, from the comparative peak areas, the different feed ratios of the multilayer films could be differentiated, with the similar crystalline peak area corresponding with PEO. The lower feed ratio of PCL exhibited the smaller crystalline peak area.

Infrared spectroscopy was also employed to confirm the chemical composition, as shown in FIG. 7. Strong absorption peaks at 1724 cm⁻¹ and 1101 cm⁻¹ corresponded to the C═O stretching band in PCL and C—O stretching band in PEO, respectively. Moreover, a tiny absorption peak at around 1607 cm⁻¹ corresponded to the aromatic band bending, which indicated the presence of the MO encapsulated guest. In fact, the MO encapsulated inside the multilayer film could be easily identified by naked eyes and quantified by UV-Vis spectroscopy.

To give a better understanding of the optical properties and especially the release kinetics of the multilayer film, a control polymer film with the same thickness (45 μm) containing only a PCL layer (layer A: PCL-PEI-b-PCL-MO, but no layer B) was fabricated by a melt compression method. The intensity of UV-Vis absorption peaked at 464 nm, due to the presence of MO, increased with the ratio of PCL in the layer because the percent of MO in the PCL layer was fixed (approximately 1% by weight). The interesting phenomenon is that the fluorescence intensity is opposite: the control polymer film [whose dyes content is highest] exhibited the lowest fluorescence intensity. This is perhaps due to the quenching effect of the dyes when they are too close with each other. Additionally, the insertion of the PEO layers between the PCL layers could decrease the fluorescence intensity to some degree. More studies on the optical properties of the dye-containing multilayer film are still under way.

### Release Kinetic Study

The multilayer polymer film with MO encapsulated inside the star-shaped copolymer was cut into small pieces (5 cm in length and 0.5 cm in width) for the release kinetic study. Taking advantage of the strong absorption peak of MO in the UV-Vis spectrum, the dye release from the layers A was recorded and calculated with predetermined time intervals.

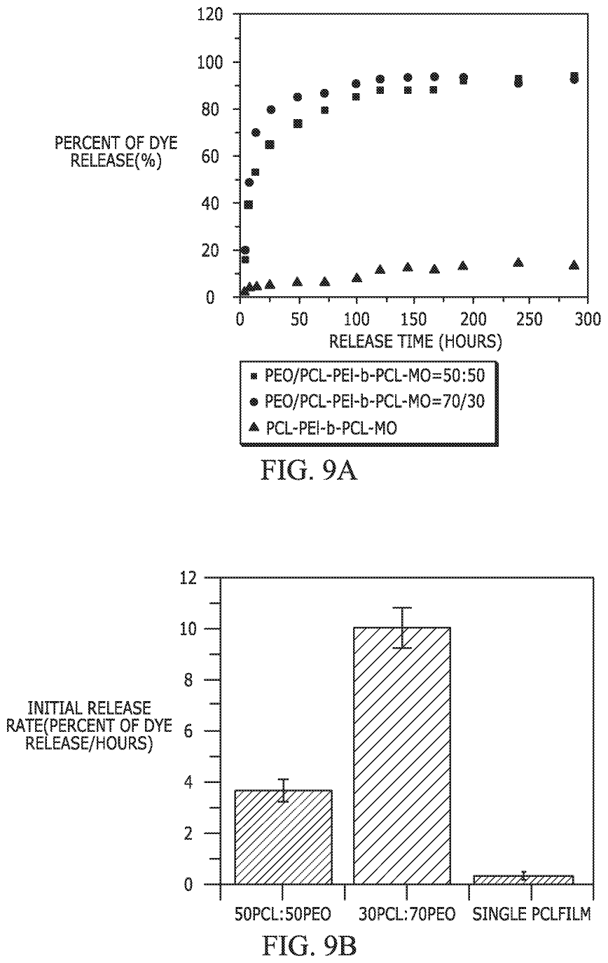

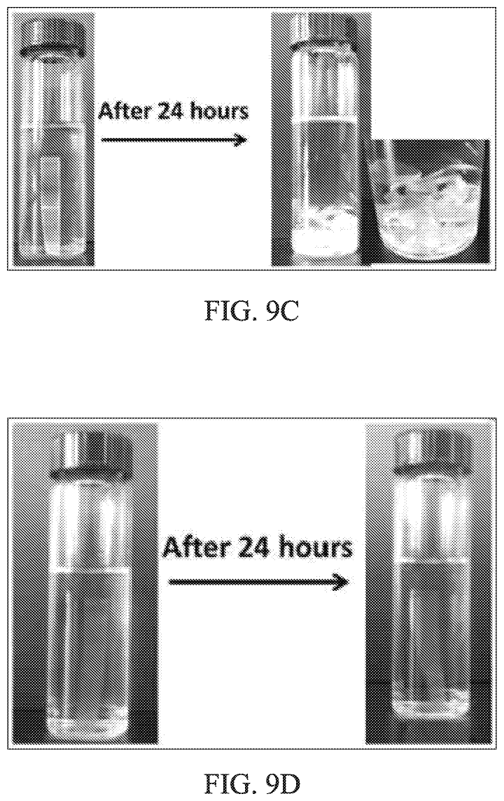

As shown in FIG. 9(a), with even the micrometer scaled thickness (45 μm), the PCL-based control film took much longer to release the guest MO due to the semi-crystalline and hydrophobic nature of PCL. The release rate was lower than 0.09% per hour. Actually, the control film released around 50% of the MO after 32 days of immersion at basic PBS solution (pH=11.0), whereas the ones in acidic and neutral PBS solution released less than 15% of MO after the same period. The multilayer film PEO/PCL-PEI-b-PCL-MO=50/50 exhibited a much higher release rate with around 70% to 80% of the MO dye being released after 2 to 3 days. The initial release rate (up to 60% release) was calculated to be 3.58±0.46% per hour.

A major difference between the appearances of the two polymer films after 24 hours of immersion in the PBS solution was shown in FIGS. 9(c) and 9(d). FIG. 9(c) is a photograph of multilayer film PEO/PCL-PEI-b-PCL-MO=50/50 in PBS solution (pH=7.4). FIG. 9(d) is a photograph of the control polymer film (PCL-PEI-b-PCL-MO) in PBS solution (pH=7.4). The control polymer film remained intact after 24 hours of immersion. In the multilayer film, however, the individual layers A, B were separated from one another.

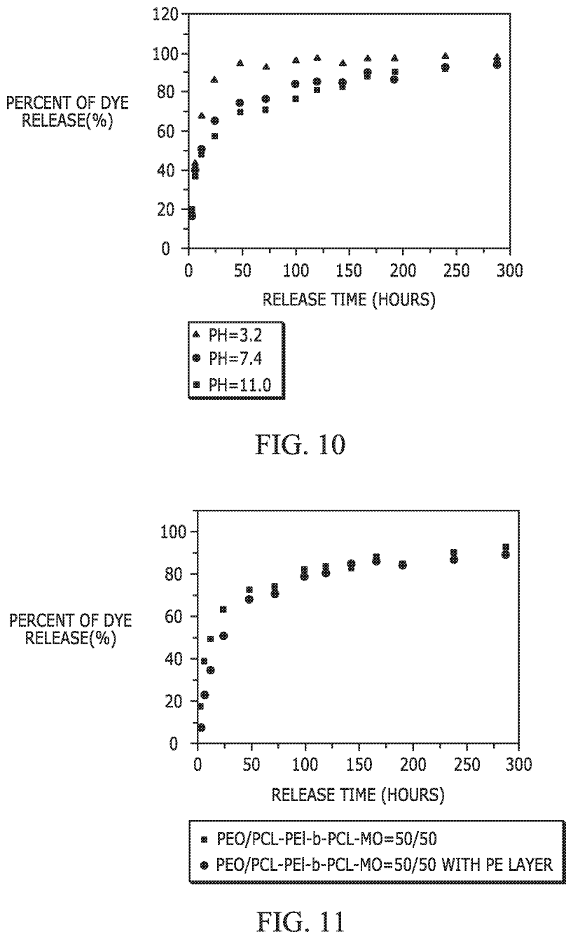

This separation process was illustrated as follows: the layers A, comprised of water-soluble PEO, were mostly dissolved in the aqueous solution after 24 hours of immersion. The layers B, comprised of water-insoluble PCL, were separated from each other and from the polymer nanosheets (layer A). The driving force for encapsulation of MO inside the star-shaped copolymer PEI-b-PCL was the electrostatic interaction between the MO and PEI core, which was largely affected by the pH values of the media. This physical affinity should also provide a method for the controlled release of encapsulated guests. The release kinetics of polymer nanosheets derived from the multilayer film PEO/PCL-PEI-b-PCL-MO=50/50 were evaluated for their pH response, as shown in FIG. 10.

Commercially available polyethylenimine (PEI) is a hyperbranched polymer composed of primary, secondary and tertiary amines. The electrostatic interaction between the PEI and the negatively charged MO is enhanced by the decrease in pH values due to the high percent of quaternization. In the same manner, the polymer nanosheet in acidic PBS solution exhibited lower guest release rate. The polymer nanosheet in basic PBS solution released much faster. The initial release rate at pH=11.0, calculated from the fitting curves, showed even six times faster than that in pH=3.2 (FIG. 10).

Forced assembly layer multiplying coextrusion could easily provide the multilayer film with the protecting layer, such as PE. The effect of the PE protecting layer on the release kinetics of the polymer nanosheet was also evaluated (see FIG. 11). Compared to the polymer nanosheet without the PE protecting layer, the one with the PE layer showed lower release rate in the PBS solution of pH=7.4 (initial release rate: 2.53±0.68% per hour vs 3.58±0.46% per hour). The relative photographs revealed the numerous free polymer nanosheets derived from the multilayer film without the PE protecting layer and restricted polymer nanosheets based on the multilayer film with the PE layer.

From the above description, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-22. (canceled)

23. A polymer construct comprising:
a multilayer polymer composite sheet comprising coextruded, alternating first and second polymer layers,
the first layers including a host polymer material and at least one amphiphilic star-shaped polymer miscible with the host polymer material, the star-shaped polymer including a polymer core and polymer branches extending therefrom to define a shell around the core, wherein a guest agent is loaded on and/or within the core, and
the second layers comprising a second polymer material substantially immiscible with the host polymer material during coextrusion,
wherein the polymer construct upon delivery to a site of interest providing controlled and/or sustained release of the guest agent upon degradation of the first polymers layers.

24. The polymer construct of claim 23, the star-shaped polymer having a hydrophilic polymer core and a hydrophobic polymer shell when provided in a water insoluble host polymer material or having a hydrophobic polymer core and a hydrophilic polymer shell when provided in a water insoluble host polymer material.

25. The polymer construct of claim 23, the host polymer material comprising a melt processable polymer that is biocompatible and, upon degradation, produces substantially non-toxic products.

26. The polymer construct of claim 23, the guest agent having a release profile from the first polymer layer at least partially defined by the degradation of the degradable host polymer material under physiological conditions.

27. The polymer construct of claim 23, the host polymer material comprising a polycaprolactone.

28. The polymer construct of claim 23, the guest agent being electrostatically coupled to the core of the star-shaped polymer.

29. The polymer construct of claim 23, the star-shaped polymer comprising an amphiphilic block copolymer.

30. The polymer construct of claim 29, the amphiphilic block copolymer comprising polyethylenimine-block-poly(caprolactone).

31. The polymer construct of claim 23 further comprising a skin layer coextruded with the first polymer layers and the second polymer layers.

32. The polymer construct of claim 23, the guest agent having a substantially homogenous distribution within the host polymer material.

33. The polymer construct of claim 23, the star-shaped polymer inhibiting diffusion of the guest agent from first polymer layers when coextruded with the second polymer layers.

34. The polymer construct of claim 23, the guest agent being a polar guest agent.

35. The polymer construct of claim 23, the guest agent being a therapeutic agent.

36. The polymer construct of claim 23, the guest agent being an imaging agent.

37. The polymer construct of claim 23, the guest agent being an anti-cancer agent.

38. The polymer construct of claim 23, the site of interest being a cell or tissue of a subject.

39. The polymer construct of claim 23 having nano-scale dimensions.

40. A microparticle comprising the polymer construct of claim 23.

41. A fiber comprising the polymer construct of claim 23.

42-59. (canceled)
